(19) United States
(12) Patent Application Publication
(10) Pub. No.: US 2003/0213493 A1
(43) Pub. Date: Nov. 20, 2003

(54) Naso-gastric tube adapter

(75) Inventor: Steven Saad, Powell, TN (US)

Correspondence Address: David T. Bracken, The Law Office of David T. Bracken, 4839 Bond Avenue, Orange, CA, 92869, US

(73) Assignee: Christopher Koo

(21) Appl. No.: 10/151,667

(22) Filed: May 20, 2002

(51) Int. Cl. A61M 16/00

(52) U.S. Cl. 128/207.18; 128/200.26

(57) ABSTRACT

The present invention is a sealing piece that can releaseably form a seal around the outside surface of a naso-gastric tube, the sealing piece being adapted to be sealingly supported from a surface having a hole at least the diameter of a distal end of a naso-gastric tube. In a specific embodiment, an elastomer sealing piece has a hole about the diameter of or just smaller than the diameter of a naso-gastric tube or other feeding tube and the sealing piece has a continuous slit from the hole to an outside edge. The slit is adapted to permit the sealing piece to be pulled apart at least enough so that the tube part of a naso-gastric tube may be sealingly located in the hole or removed from it. In operation, the invention would permit the patient having an appropriately inserted naso-gastric tube to threat the distal end of the naso-gastric tube to an access location external to a pressurized ventilation environment, i.e., a ventilation or anesthesia mask and associated gas tubing and access ports.

### BACKGROUND OF THE INVENTION

The present invention relates to devices permitting ventilation of a human patient who has had a naso-gastric tube inserted through the nasal cavity through to the stomach.

The prior art has attempted to maintain external access to an in-place naso-gastric tube in a number of ways. U.S. Pat. No. 5,474,060 describes respiratory face masks for administering a gas to a patient when an operation is performed involving a local anaesthetic or when a patient is recuperating in an intensive care unit or post-anaesthetic care unit. The face mask includes an inlet for directing a flow of gas to the interior of the mask, and a port for allowing the exhaled air stream by the patient to flow therethrough, and to a tube for directing such flow to a mass spectrometer or capnograph. The port includes a connector having a configuration for releasably engaging one end of the tube presented thereto.

U.S. Pat. No. 4,328,797 describes a mask for delivery of gas, such as oxygen, to a patient and useable in a naso-gastric intubation procedure without disruption of the seal between the mask and the face of the patient. The body of the mask includes a fenestration or opening through which the naso-gastric tube may be inserted. The fenestration is preferably shaped to support the tube in manner which is comfortable for the patient and which eliminates or minimizes the amount of gas lost to the atmosphere outside the mask. In at least some embodiments, the fenestration is closed when a tube is not inserted therethrough so that the mask may be used normally without loss of the gas through the fenestration. However, the fenestration is located at the sealing edge of the mask to be pressed against the user's face, whereby a relatively large slot must extend from the mask edge to a hole in the support shell of the mask. Such a concept is incapable of being used in modern respiration masks with inflatable cuffs at the sealing edge of the mask. No such slot may be maintained in the continuous inflatable cuffs of the current respiration masks. In addition, the sealing means at the fenestration for sealing the hydraulic circuit of patient respiration is unlikely to be adequately maintained, as it appears the sealing means is a simple and relatively thin rubber sheet with a slit in it. After a naso-gastric tube is located in the slit for some time, the “memory” response of the slit is impaired and the slit will remain slightly apart upon removal of the naso-gastric tube from the fenestration. Thus, the teaching of this patent is a failure in the realm of long term sealing means for a naso-gastric tube.

U.S. Pat. No. 3,730,179 shows a naso-tube that passes through a housing for an endotracheal tube. The transition piece of the tube through the housing is a simple friction fit gasket. This friction fit gasket is shown in a somewhat larger diameter scale in U.S. Pat. No. 3,388,705, where the primary air supply conduit is connected to a substantially normal surface to the insertion part of the conduit.

The prior art describes a problem in preserving the naso-gastric tube placement, inwardly radial pressure for sealing to a support housing and external access during periods of respiration through a face mask. However, a workable device for long term care is not disclosed in the prior art, except indicating the need for such a device.

### SUMMARY OF THE INVENTION

The present invention is a sealing piece that can releaseably form a seal around the outside surface of a naso-gastric tube, the sealing piece being adapted to be sealingly supported from a surface having a hole at least the diameter of a distal end of a nasogastric tube. In a specific embodiment, an elastomer sealing piece has a hole about the diameter of or just smaller than the diameter of a naso-gastric tube or other feeding tube and the sealing piece has a continuous slit from the hole to an outside edge. The slit is adapted to permit the sealing piece to be pulled apart at least enough so that the tube part of a naso-gastric tube may be sealingly located in the hole or removed from it. In operation, the invention would permit the patient having an appropriately inserted naso-gastric tube to threat the distal end of the naso-gastric tube to an access location external to a pressurized ventilation environment, i.e., a ventilation or anesthesia mask and associated gas tubing and access ports. Thus, the naso-gastric tube would not have to be removed for long term ventilation care as in comatose patients. Even the unskilled person will appreciate that insertion, removal and re-insertion of a naso-gastric tube introduces measurable trauma to a patient and drags to the mouth and airway some part of the contents of the stomach.

There is a need for a means for retaining the function and location of the naso-gastric tube that is inexpensive and easily inserted into the hydraulic circuit needed for ventilation or anesthesia.

### BRIEF DESCRIPTION OF THE DRAWINGS

FIG. 1 is a view of the prior art device of U.S. Pat. No. 4,328,797.

FIG. 2 is a perspective view of a modern respiration mask without its inflatable cuff.

FIG. 3 is a side view of an embodiment of the invention showing the L-shape of a support case for insertion in the hydraulic circuit for patient respiration.

FIG. 4 is the device of FIG. 3 with a naso-gastric tube sealingly secured in place.

FIG. 5 is a perspective and cut away view of the invention sealing means with an exemplary naso-gastric tube.

FIG. 6 is a top view of the invention sealing means with an exemplary naso-gastric tube sealingly secured in it.

FIG. 7 is the view of FIG. 6 where the invention sealing means are caused to be spread apart at a slit to allow removal or insertion of the naso-gastric tube to sealing hole for sealing securement.

FIG. 8 is an alternate embodiment of the sealing means of FIG. 6 showing a much reduced outer diameter.

FIG. 9 is a side cut away view of the sealing means of FIG. 6 with a plug in place to cap the sealing means when the naso-gastric tube is removed.

FIG. 10 is section AA of FIG. 3.

FIG. 11 is section BB of FIG. 4.

FIG. 12 is the mask of FIG. 2 with an opening adapted to permit sealing insertion of a support case and sealing means.

FIG. 13 is section CC of FIG. 12 with side cut away view of an alternate embodiment of the invention inserted into the hole in the support shell of the mask.

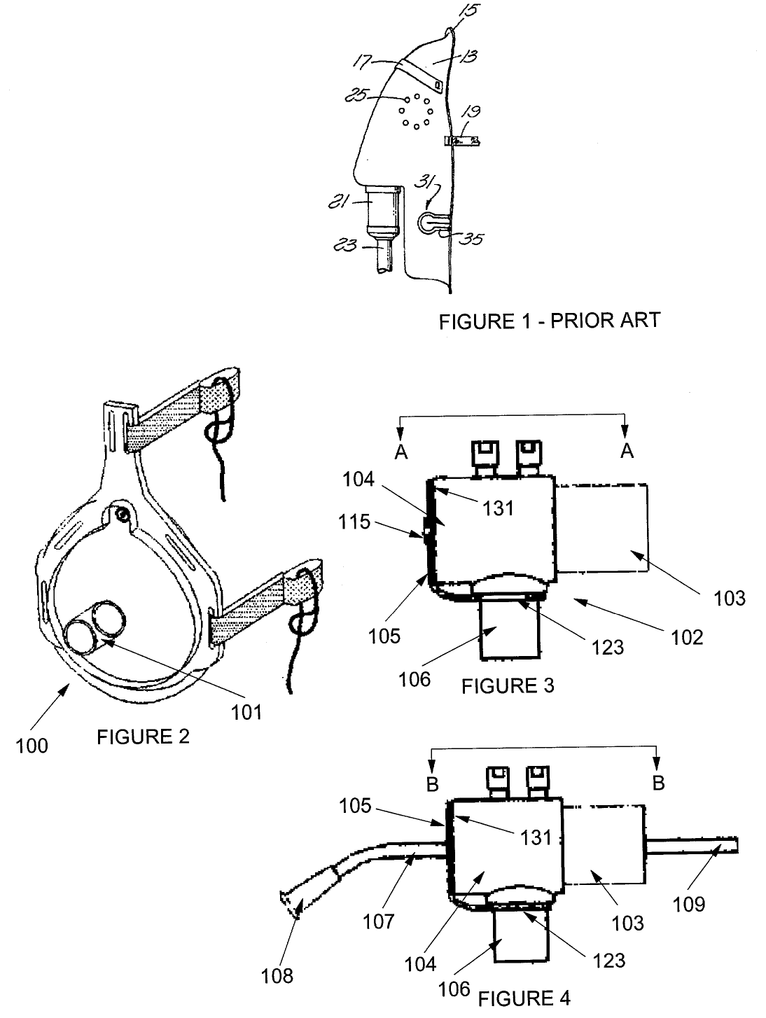

### DETAILED DESCRIPTION OF THE INVENTION

The present invention uses an assembly of a support case with sealing means to be inserted or given access to the hydraulic circuit for respiration of a patient. The assembly allows external access and injection or withdrawal of fluids to the patient's stomach while pressurized respiration is taking place. A sealed hole is formed in the support case at least large enough for the end of the of the naso-gastric tube to be passed through it. When the end of the naso-gastric tube is passed through structure intervening between the patients nostrils and through that sealed hole of the support case, the sealing means are opened along a slit from an outside edge to a naso-gastric tube hole, the naso-gastric tube is located in the naso-gastric tube hole, and the slit in the sealing means is brought back together to seal the naso-gastric tube in the nasogastric tube hole, whereafter the sealing means are located in the sealed hole of the support case. The support case is adapted to sealingly connect with the hydraulic circuit so that no significant gas volume is lost by application of the invention to hydraulic circuit.

The prior art often attempts to solve the problem of providing a transition piece that will seal a housing with the septum type device shown in the helmet port of U.S. Pat. No. 3,067,425. Such a transition would be inappropriate for a naso-gastric tube. The soft, small diameter naso-gastric tubes cannot be pressed through such a transition and substantial pressure on the outside surface of the tube will cause it to constrict, severely impairing the ability to deliver many times viscous liquids to a patients stomach. It is well known that naso-gastric tubes are used to extract stomach contents to determine the progress of digestion, essentially eliminating sealing means that put substantial inwardly radial pressure on the tube to seal it to a support housing. U.S. Pat. No. 6,273,087 points out that difficulty for air sampling by forming a transition from the exterior of a rigid housing of the same rigid material as the housing. Reducing the hydraulic diameter of the naso-gastric tube by more than about 20 percent would be an unacceptable level of occlusion if such a tube were to be sealed to a support housing by a prior art device, which occlusion appears to occur with such devices.

The present invention preferable uses a short, spool shaped piece of an elastomeric polymer or rubber so that the friction coefficient between it and the naso-gastric tube is sufficiently high so that only light pressure is required to seal the tube to the adapter and hold it in place. This type of light pressure is only achievable with the slitted access to the bore of the adapter.

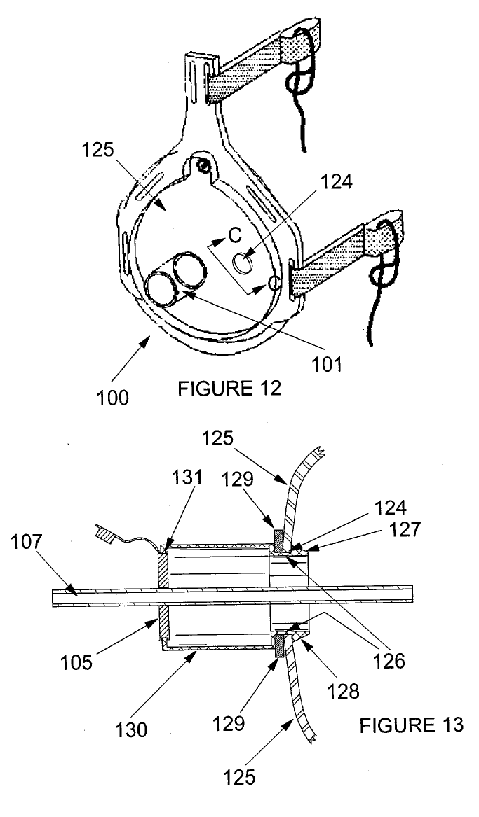

In one preferred embodiment of the invention as in FIGS. 3-11, the invention assembly has a support case adapted to be inserted between a respirator hose and the mask hose connector or between sections of the respirator hose. In another preferred embodiment as in FIGS. 12 and 13, the invention assembly has a support case adapted to be inserted in the mask support shell, the hose sections or support or transition pieces for those hose sections. So the invention assembly may be effectively inserted or located at almost any location in the hydraulic circuit established for respiration of a patient.

FIG. 2 shows a generalized view of a modern mask 100 with hose connector 101 but without its inflatable cuff. The invention assembly of FIG. 3 shows that the support case 102 comprises connectors 103 and 106 adapted to connect with respirator hose connectors and/or connector 101, although connector 103 is preferably adapted to be connected with connector 101 to locate the case 102 nearest to the nostril access of naso-gastric tube 107 into the patient. Connectors 103 and 106 extend preferably in an L-shape from section 104. Section 104 defines sealed hole 131 into which is sealingly fitted sealing means 105. FIGS. 3 and 4 show sealing means 105 with an optional extension forming loop 123 to fit around the outer surface of connector 106 so sealing means 105 will not fall away from the invention assembly in the process of manipulating the naso-gastric tube 107 into and out of sealing relationship with the sealing means 105.

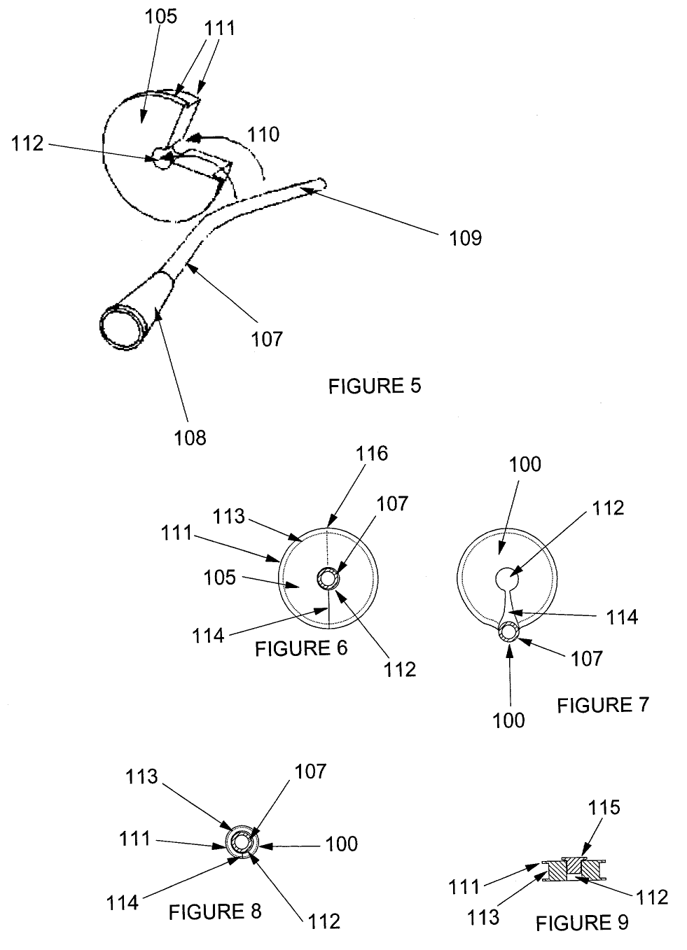

FIG. 3 shows sealing means 105 with its outer edge in sealing contact with the edges of hole 131, with a plug 115 in the naso-gastric tube hole 112. FIG. 4 shows sealing means with plug 115 removed and replaced, via the action 110 shown in FIG. 5, with a portion of the naso-gastric tube 107. FIG. 4 shows that tube 107 extends from an external end 108 to a hydraulic circuit portion 109, the intervening tube portion being sealed at some part of its outside surface by the sealing means 105. Thus, when connectors 103 and 106 are connected to the respiration hydraulic circuit, end 108 is accessible for external injection of fluids while respiratory actions are accomplished without interference from the naso-gastric tube 107 or gas loss from the invention assembly.

Sealing means 105 preferably has an outer edge with adaptation to sealing mate to the hole 131 in section 104. A preferred form of these adaptations are shown in FIGS. 5 and 9, with the edge having lips 111 extending further from the piece center than the notch or groove portion 113. Thus, section 104 may be made of a relatively rigid plastic or metal while sealing means 105 is made of a deformable elastomer that is capable of accomplishing the sealing functions of the invention while being deformable as shown in FIG. 7 along slit 114 to open sufficiently to allow sideways insertion of naso-gastric tube 107 through the slit 114 into and out of hole 112. FIG. 6 shows that slit 112 may be extended beyond hole 112 along line 116, although such an embodiment is less preferable. In an alternate embodiment of the sealing means, slit 112 is extended completely across the sealing means 105 along line 116 so that that two pieces are formed and are adapted to be mated or joined together about naso-gastric tube 107 to form the invention naso-gastric tube hole 112.

FIG. 8 shows an alternate embodiment of the present invention, whereby the sealing means 105 is reduced to an effective diameter just sufficient to enable sealing of means 105 to the sealed hole 131 and the additional required sealing of means 105 to naso-gastric tube 107. Sealing means 105 is made so that when the naso-gastric tube 107 is located in naso-gastric tube hole 112, then the combination is insertable into sealed hole 131 for sealing connection of means 105 thereto.

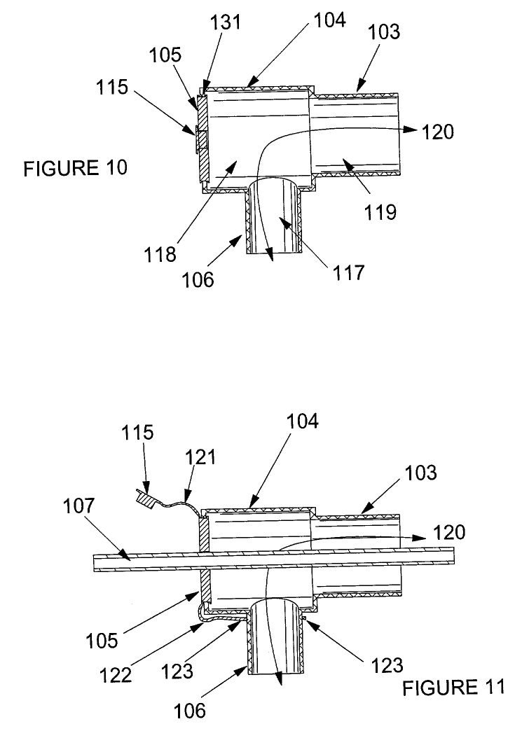

The present invention of FIGS. 3 and 4 are shown in cut away side views FIGS. 10 and 11 without and with the naso-gastric tube 107 secured in the invention assembly. It is readily appreciated that gas flow 120 is not significantly affected by the location of naso-gastric tube 107 in its hydraulic path. Support case 102 is seen in FIGS. 10 and 11 as being a shell adequate for effective passage of gas flow 120 through the invention assembly.

However, the invention also comprises in an alternate embodiment as in FIGS. 12 and 13 so that the L-shaped connector of support case 102 in the hydraulic circuit might be avoided. For this embodiment, a hole 124 is formed the support shell 125 of mask 100. Support case 130 now just comprises a hollow shell with a sealed hole 131 for sealing means 105 as in the previous embodiments extending to an opening defined by wall 126 having on their outside distal rim locking notches 127. The outer diameter of wall 126 is adapted to be about the diameter of hole 124 and has located about it gasket 129. When the notched end of wall 126 is inserted into hole 124 with gasket 129 pressed between the outside surface of support shell 124, support case 130 is sealed to the hydraulic circuit for pressured respiration and at the same time providing external access to tube 107 as in the previous embodiments. Thus, the invention assemblies comprise means for inserting into the hydraulic circuit a support case through which flows the entire gas volumes for respiration (as in FIG. 11) or they also comprise means for locating the support case so that substantially no gas volumes of respiration pass through the support case (as in FIG. 13).

Composition of the sealing means may be of rubber or synthetic elastomers, at least at the sealing surfaces to sealed hole 131 and around tube 107. It is within the scope of the invention to form an intervening portion between those sealing surface with other materials that will permit opening or deformation along a slit for passage of tube 107 a hole 112.

The above design options will sometimes present the skilled designer with considerable and wide ranges from which to choose appropriate apparatus and method modifications for the above examples. However, the objects of the present invention will still be obtained by that skilled designer applying such design options in an appropriate manner.

I claim:

1. A combination of a naso-gastric tube, a tube adapter and a substantially rigid support housing comprising:
(a) the support housing having a main bore with two ends, where a first end is sealingly connected to a first conduit adapted to deliver air from a respirator into the bore and a second end is sealingly connected to a second conduit adapted to connect with a respirator mask for delivery of air from the bore to the respirator mask;
(b) the support housing further defining an adapter opening from the outside of the support housing to the inside of the bore;
(c) the tube adapter having a flattened spool shape having a lateral surface that sealingly engages the support housing bore;
(d) the tube adapter further defining an axially parallel bore and a slit extending laterally from the entire length of the axially parallel bore to an outside surface of the tube adapter, so that finger pressure is capable of repeatedly opening the tube adapter along the slit without breaking the tube adapter to expose the length of the axially parallel bore and thereafter an engaged part of the naso-gastric tube is capable of being located in the axially parallel bore, whereby when the finger pressure is removed and the engaged part of the naso-gastric tube in the axially parallel bore is sealingly engaged therein; and
(e) the engaged part of the naso-gastric tube is distal to a stomach end and is sealingly engaged in the axially parallel bore, such that a part of the nasogastric in the direction of the stomach end extends through the support housing bore into the second conduit.

2. The combination of claim 1 wherein the engaged part is sealingly engaged with the axially parallel bore without occluding more than about 20 percent of the hydraulic diameter of the naso-gastric tube.

3. The combination of claim 1 wherein a part of the naso-gastric tube more distal to the stomach end than the engaged part extends outside of the support housing to an injection fitting.

4. The combination of claim 1 wherein the spool shape of the tube adapter comprises two ends with circumferential ridges about them adapted to maintain the tube adapter in the adapter opening.

5. The combination of claim 1 wherein the tube adapter consists of an elastomeric polymer with a friction coefficient close to that of natural rubber.

6. The combination of claim 1 wherein the tube adapter consists of natural rubber or a flexible mixture of rubber and other synthetic polymers.

7. The combination of claim 1 wherein the naso-gastric tube has an outside diameter of about less than four millimeters.

8. The combination of claim 1 wherein the support housing defines the bore with about a right angle change in axial direction from the first end to the second end.

9. The combination of claim 8 wherein an axis of the adapter opening is about the same as an axis of the second opening.

10. A combination of a naso-gastric tube, a tube adapter and a substantially rigid support housing comprising:
(a) the support housing having a main bore with two ends, where a first end is an adapter opening and the second end is sealingly connected to a respirator mask shell, such that the adapter opening extends from the outside of the support housing to the inside of the bore;
(b) the tube adapter having a flattened spool shape having a lateral surface that sealingly engages the support housing bore;
(c) the tube adapter further defining an axially parallel bore and a slit extending laterally from the entire length of the axially parallel bore to an outside surface of the tube adapter, so that finger pressure is capable of repeatedly opening the tube adapter along the slit without breaking the tube adapter to expose the length of the axially parallel bore and thereafter an engaged part of the naso-gastric tube is capable of being located in the axially parallel bore, whereby when the finger pressure is removed and the engaged part of the naso-gastric tube in the axially parallel bore is sealingly engaged therein; and
(d) the engaged part of the naso-gastric tube is distal to a stomach end and is sealingly engaged in the axially parallel bore, such that a part of the nasogastric in the direction of the stomach end extends through the support housing bore into the inside of the respirator mask shell.

11. The combination of claim 10 wherein the engaged part is sealingly engaged with the axially parallel bore without occluding more than about 20 percent of the hydraulic diameter of the naso-gastric tube.

12. The combination of claim 10 wherein a part of the naso-gastric tube more distal to the stomach end than the engaged part extends outside of the support housing to an injection fitting.

13. The combination of claim 10 wherein the spool shape of the tube adapter comprises two ends with circumferential ridges about them adapted to maintain the tube adapter in the adapter opening.

14. The combination of claim 10 wherein the tube adapter consists of an elastomeric polymer with a friction coefficient close to that of natural rubber.

15. The combination of claim 10 wherein the tube adapter consists of natural rubber or a flexible mixture of rubber and other synthetic polymers.

16. The combination of claim 10 wherein the naso-gastric tube has an outside diameter of about less than four millimeters.
